(19) United States
(12) Patent Application Publication
(10) Pub. No.: US 2022/0339429 A1
(43) Pub. Date: Oct. 27, 2022

(54) BONE STIMULATOR AND BONE STIMULATION SYSTEM FOR BONE FRACTURE HEALING

(71) Applicant: THE HONG KONG POLYTECHNIC UNIVERSITY, Hong Kong (CN)

(72) Inventors: Shuai LI, Hong Kong (CN); Md. Monzurul ALAM, Hong Kong (CN); Yongping ZHENG, Hong Kong (CN); Poornima PALANISAMY, Hong Kong (CN)

(21) Appl. No.: 17/753,689

(22) PCT Filed: Sep. 16, 2020
(86) PCT No.: PCT/CN2020/115535

§ 371 Date: Mar. 11, 2022

Related U.S. Application Data
(60) Provisional application No. 62/900,689, filed on Sep. 16, 2019.

(51) Int. Cl. A61N 1/04; A61B 17/72

(52) U.S. Cl. CPC A61N 1/04; A61B 17/72; A61B 2017/0011

(57) ABSTRACT

A bone stimulator (30,40,50,610,810,910), a bone stimulation system (200,600,800, 900) and method for bone fracture healing of a broken bone (930,830,630,55) in a body, which can speed up the healing rate. Accordingly, ultrasound (214) is used by the present system to power up the implanted bone stimulator (30,40,50,610,810, 910) for generating a stimulating electric current passing through a broken area (931, 831,631,56) in the broken bone (930,830,630,55) for bone fracture healing.

### TECHNICAL FIELD

The present disclosure generally relates to a bone stimulator and a bone stimulation system for bone fracture healing.

### BACKGROUND

Fracture, also known as broken bone, is a condition that changes the shape of the bone. Bone fractures are common in human and can be caused by trauma such as sports injuries and car accidents, or osteoporosis. As bones render a frame to support the human body, it is essential to heal the bone as quickly as possible. Once a bone fracture happens, giving the fixture may be a prevailing method. However, the rate of bone healing differs from person to person because of the patients' age, types of bone fracture, site of injury and some biological processes. Additionally, insufficient treatment of bone after severe fracture leads to many complications including bone weakness, abnormal healing and loss of functions. Hence, it is essential to find an efficient method to treat the bone fractures.

Healing of fractured bone by using electric current has been reported. Few clinical studies demonstrated that electrical stimulation techniques are not just effectual to accelerate bone growth, but also have the ability to reduce the pain. Conventionally, the electrical stimulation can be generated and applied to bones by the following techniques: capacitive coupling stimulation and direct current electrical stimulation.

Capacitive coupling (CC) is well known for its non-invasive characteristic where two cutaneous electrodes are placed over the skin on the opposite region of the site and generate the electric field. As shown in FIG. 1A, a pair of electrodes is placed opposite to each other near the fracture site and an external power source is used to generate the current. CC works on the mechanism that calcium voltage-gated channels are activated which results in calcium translocation to increase the cell proliferative response. The upregulation of calcium and growth factors induces bone formation. During CC stimulation, the electric potential of 1-10V at a frequency of 20-200 kHz is applied to produce an electric field of 1-100 mV/cm in the tissue between the two capacitor plates. However, bone has a higher impedance resulting a lower current passing through the targeted area and it requires quite a long treatment period. In addition, the healing rate can also be significantly affected by the placement of capacitor plates and the size of the limb. The capacitor plates also lead to other problems including the restriction of patient's daily activity due to the wires connection with an external power source and if the electrode pads are placed too close to each other, it may cause skin irritation and allergic reaction to the patients.

Direct current stimulation (DCS) is effective but invasive method in which one or more cathodes are implanted close to the site to repair. As shown in FIG. 1B, cathodes are placed near the injured site and an electrode is placed at a distant site. An external or internal power source is used to generate the current. The possible mechanism of DCS includes the faradic reaction at cathode that helps to increase the number of osteoinductive factors which play a major role in bone formation. Usually, in DCS the cathode is placed at the area of injury so that it covers maximum area for stimulation near fracture site, and the anode is placed near the soft tissue to allow 5-100 μA of direct current. Although the advantage of the implantable device is that it does interface the daily activities of the patient as the whole system is placed under the skin, there are several disadvantages exist. The device along with battery makes the equipment quite larger in size which makes the implantation more difficult and cause soft tissue discomfort. Furthermore, a battery replacement surgery, a consequence of limited battery life and no existing wireless charging, there are more risks for infection for the second surgery. Other limitation of this DC stimulation includes electrode placement. Since typical wire electrodes are used as cathodes, some displacement of the electrodes happen during the healing period. Sometimes short-circuiting to the anode may occur when multiple cathodes are implanted. Furthermore, the removal of the cathode may become impossible due to the bone growth during the healing process which can also cause infections. Improper placement of electrodes also affects bone formation. When the cathode and anode are placed too proximally, bone failed to heal. To achieve the better result, the cathode is required to be placed 5 cm in distance from the anode.

A need therefore exists for an improved apparatus and method for bone fracture healing that eliminates or at least diminishes the disadvantages and problems described above.

### SUMMARY OF THE INVENTION

Certain embodiments of the present disclosure provide a bone stimulator for bone fracture healing of a broken bone in a body, the bone stimulator being implantable in the body and comprising: a piezoelectric transducer for converting power of ultrasound into electric power; a signal conditioning circuit for generating a stimulating electric current from the electric power; a first stimulating electrode for contacting the broken bone or being located adjacent to the broken bone; and a second stimulating electrode for contacting the broken bone or being located adjacent to the broken bone, the first stimulating electrode and the second stimulating electrode being arranged such that the broken area in the broken bone is located between the first stimulating electrode and the second stimulating electrode such that the stimulating electric current passes through the broken area.

In certain embodiments, the first stimulating electrode comprises a bone fixation component for contacting the broken bone and fixing the broken bone in place.

Certain embodiments of the present disclosure provide a bone stimulation system for bone fracture healing of a broken bone comprising: the bone stimulator described above; and a bone fixation component for fixing the broken bone in place, the first stimulating electrode being used for being attached to the first bone fixation component.

Certain embodiments of the present disclosure provide a bone stimulation system for bone fracture healing of a broken bone comprising: the bone stimulator described above; and a bone fixation structure comprising a bone fixation plate and a first bone fixation component, the bone fixation plate being used for being attached to the broken bone for fixing the broken bone in place, the first bone fixation component being used for connecting the bone fixation plate to the broken bone, the first stimulating electrode being used for being attached to the first bone fixation component.

Certain embodiments of the present disclosure provide a bone stimulation method for bone fracture healing of a broken bone in a body comprising: providing the bone stimulator described above, the bone stimulator being implanted in the body such that the first stimulating electrode contacts the broken bone or is located adjacent to the broken bone, the second stimulating electrode contacts the broken bone or is located adjacent to the bone broken, the broken area is located between the first stimulating electrode and the second stimulating electrode; and generating ultrasound toward the piezoelectric transducer via the skin of the body such that the stimulating electric current is generated and passes through the broken area.

Certain embodiments of the present disclosure provide a bone stimulation method for bone fracture healing of a broken bone in a body comprising: providing the bone stimulator described above, the bone stimulator being implanted in the body such that the first stimulating electrode contacts the broken bone or is located adjacent to the broken bone, the second stimulating electrode contacts the broken bone or is located adjacent to the bone broken, the broken area is located between the first stimulating electrode and the second stimulating electrode; and generating ultrasound toward the broken area and the piezoelectric transducer via the skin of the body such that the broken area is stimulated by the ultrasound and the stimulating electric current is generated and passes through the broken area.

Certain embodiments of the present disclosure provide a bone stimulation method for bone fracture healing of a broken bone in a body comprising: providing the bone stimulation system described above, the bone stimulator and the bone fixation component being implanted in the body such that the first stimulating electrode contacts the broken bone, the second stimulating electrode contacts the broken bone or tissue adjacent to the broken bone, the broken area is located between the first stimulating electrode and the second stimulating electrode; and generating ultrasound toward the piezoelectric transducer via the skin of the body such that the stimulating electric current is generated and passes through the broken area of the bone fracture.

Certain embodiments of the present disclosure provide a bone stimulation method for bone fracture healing of a broken bone in a body comprising: providing the bone stimulation system described above; the bone stimulator and the bone fixation structure being implanted in the body such that the first stimulating electrode contacts the broken bone, the second stimulating electrode contacts the broken bone, the broken area is located between the first stimulating electrode and the second stimulating electrode; and generating ultrasound toward the piezoelectric transducer via the skin of the body such that the stimulating electric current is generated and passes through the broken area.

Certain embodiments of the present disclosure provide a bone stimulation method for bone fracture healing of a broken bone in a body comprising: generating ultrasound; converting power of the ultrasound into electric power; generating a stimulating electric current from the electric power; and passing the stimulating electric current through the broken area in the broken bone.

Certain embodiments of the present disclosure provide a bone stimulator for bone fracture healing of a broken bone in a body, the bone stimulator being implantable in the body and comprising: a piezoelectric transducer for converting mechanical power into electric power; a signal conditioning circuit for generating a stimulating electric current from the electric power; a first stimulating electrode for contacting or being located adjacent to the broken bone; and a second stimulating electrode for contacting the broken bone or being located adjacent to the broken bone, the first stimulating electrode and the second stimulating electrode being arranged such that the broken area in the broken bone is located between the first stimulating electrode and the second stimulating electrode such that the stimulating electric current passes through the broken area.

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Other aspects of the present invention are disclosed as illustrated by the embodiments hereinafter.

### BRIEF DESCRIPTION OF DRAWINGS

The appended drawings, where like reference numerals refer to identical or functionally similar elements, contain figures of certain embodiments to further illustrate and clarify the above and other aspects, advantages and features of the present invention. It will be appreciated that these drawings depict embodiments of the invention and are not intended to limit its scope. The invention will be described and explained with additional specificity and detail through the use of the accompanying drawings in which:

FIG. 1A depicts capacitive coupling stimulation of the prior art;

FIG. 1B depicts direct current stimulation of the prior art;

FIG. 2 depicts a bone stimulation system for bone fracture healing according to certain embodiments;

FIG. 3 depicts a bone stimulator according to certain embodiments;

FIG. 4 depicts a screw-typed bone stimulator according to certain embodiments;

FIG. 5 depicts a screw-typed bone stimulator according to certain embodiments;

FIG. 6 depicts a bone stimulation system having a non-metallic screw according to certain embodiments;

FIG. 7 depicts a bone fixation component according to certain embodiments;

FIG. 8 depicts a bone stimulation system having a bone fixation plate, two coiled cathodes and a wire anode according to certain embodiments;

FIG. 9 depicts a bone stimulation system having a bone fixation plate and coiled electrodes according to certain embodiments;

FIG. 10 depicts a bone fixation plate according to certain embodiments;

FIG. 11 depicts an acoustic absorber according to certain embodiments;

FIG. 12 an ultrasonic generator embedded in a wearable protective gear according to certain embodiments;

FIG. 13A depicts an experimental set-up for measuring a stimulating electric current generated by a bone stimulator according to certain embodiments;

FIG. 13B shows output direct current of the bone stimulator at different ultrasound intensities;

FIG. 14 is a flow chart depicting a bone stimulation method according to certain embodiments;

FIG. 15 is a flow chart depicting a bone stimulation method according to certain embodiments;

FIG. 16 is a flow chart depicting a bone stimulation according to certain embodiments; and

FIG. 17 is a flow chart depicting a bone stimulation method according to certain embodiments.

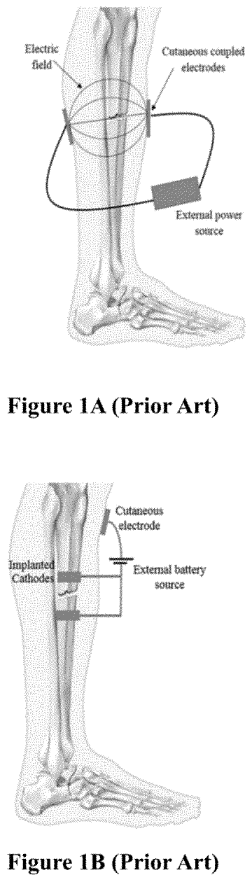

Skilled artisans will appreciate that elements in the figures are illustrated for simplicity and clarity and have not necessarily been depicted to scale.

### DETAILED DESCRIPTION OF THE INVENTION

The present disclosure provides a bone stimulator, a bone stimulation system and method for bone fracture healing of a broken bone in a body, which can speed up the healing rate and even can repair the delayed union and nonunion. Accordingly, ultrasound is used by the present system to power up the implanted bone stimulator for generating a stimulating electric current passing through a broken area in the broken bone for bone fracture healing. In addition, the bone stimulator can be combined with the bone fixation component such that extra surgery for removing the bone stimulator can be avoided. The combining effect from both electrical bone stimulation and ultrasound bone stimulation is also obtained by the present method.

Certain embodiments of the present disclosure provide a bone stimulator for bone fracture healing of a broken bone in a body, the bone stimulator being implantable in the body and comprising: a piezoelectric transducer for converting power of ultrasound into electric power; a signal conditioning circuit for generating a stimulating electric current from the electric power; a first stimulating electrode for contacting the broken bone or being located adjacent to the broken bone; and a second stimulating electrode for contacting the broken bone or being located adjacent to the broken bone, the first stimulating electrode and the second stimulating electrode being arranged such that the broken area in the broken bone is located between the first stimulating electrode and the second stimulating electrode such that the stimulating electric current passes through the broken area.

In certain embodiments, the piezoelectric transducer, the signal conditioning circuit, the first stimulating electrode and the second stimulating electrode are biocompatible.

In certain embodiments, the piezoelectric transducer, the signal conditioning circuit, the first stimulating electrode and the second stimulating electrode are biodegradable.

In certain embodiments, the piezoelectric transducer comprises a polymeric piezoelectric material or an inorganic piezoelectric material.

In certain embodiments, the piezoelectric transducer comprises lead zirconate titanate (Pb[ZrₓTi₁₋ₓ]O₃), lead titanate (PbTiO₃), Zinc oxide (ZnO), barium titanate (BaTiO₃) or polyvinylidene difluoride (PVDF).

In certain embodiments, each of the first stimulating electrode and the second stimulating electrode comprises copper, titanium, silver, or a carbon-based material.

In certain embodiments, the bone stimulator further comprises a coating layer or an enclosure for protecting the piezoelectric transducer and the signal conditioning circuit.

In certain embodiments, the coating layer and the enclosure are biocompatible or biodegradable.

In certain embodiments, the coating layer and the enclosure comprise silicone, polytetrafluoroethylene, polydimethylsiloxane (PDMS), dimethyl silicone, or polyurethane.

In certain embodiments, the first stimulating electrode comprises a bone fixation component for contacting the broken bone and fixing the broken bone in place.

In certain embodiments, the bone fixation component is used for being inserted into the broken bone through the broken area.

In certain embodiments, the bone fixation component includes a screw, a pin, a nail, a rod, a panel or a plate.

In certain embodiments, the bone fixation component comprises a metallic material, a conductive biodegradable material, a conductive polymeric material, or a conductive ceramic material.

In certain embodiments, the bone fixation component comprises a hole, the piezoelectric transducer and the signal conditioning circuit being accommodated within the hole.

In certain embodiments, the bone stimulator further comprises a coating layer closing the hole.

In certain embodiments, the first stimulating electrode further comprises a connecting portion connecting the bone fixation component to the signal conditioning circuit.

In certain embodiments, the first stimulating electrode comprises a first bone fixation component for fixing the broken bone in place; and the second stimulating electrode comprises a second bone fixation component for fixing the broken bone in place.

In certain embodiments, the stimulating electric current is direct current in a range of 1 μA to 30 mA.

In certain embodiments, the waveform and the magnitude of the stimulating electric current is controlled by an external ultrasound generator.

In certain embodiments, the waveform is sinusoidal, pulse, square wave, triangle wave, random noise or music.

Certain embodiments of the present disclosure provide a bone stimulation system for bone fracture healing of a broken bone comprising: the bone stimulator described above; and a bone fixation component for fixing the broken bone in place, the first stimulating electrode being used for being attached to the first bone fixation component.

In certain embodiments, the bone fixation component is electrically non-conductive.

In certain embodiments, the bone fixation component comprises polyglycolide, polylactide or polylactic acid-polyglycolic acid copolymer.

In certain embodiments, the first stimulating electrode coils around the bone fixation component.

In certain embodiments, the bone stimulation system further comprises an ultrasound generator for generating the ultrasound toward the piezoelectric transducer, or toward the piezoelectric transducer and the broken area.

In certain embodiments, the ultrasound generator is configured to generate the ultrasound having a frequency between 0.5 MHz and 20 MHz and an ultrasound intensity between 1 mW/cm² and 3 W/cm².

Certain embodiments of the present disclosure provide a bone stimulation system for bone fracture healing of a broken bone comprising: the bone stimulator described above; and a bone fixation structure comprising a bone fixation plate and a first bone fixation component, the bone fixation plate being used for being attached to the broken bone for fixing the broken bone in place, the first bone fixation component being used for connecting the bone fixation plate to the broken bone, the first stimulating electrode being used for being attached to the first bone fixation component.

In certain embodiments, the bone fixation structure further comprises a second bone fixation component for connecting the bone fixation plate to the broken bone, the second stimulating electrode being used for being attached to the second bone fixation component.

In certain embodiments, the bone fixation plate comprises a hole, the piezoelectric transducer and the signal conditioning circuit being accommodated in the hole.

In certain embodiments, the bone stimulation system further comprises a coating layer closing the hole.

In certain embodiments, the bone fixation plate comprises stainless steel, pure titanium or titanium alloy.

Certain embodiments of the present disclosure provide a bone stimulation method for bone fracture healing of a broken bone in a body comprising: providing the bone stimulator described above, the bone stimulator being implanted in the body such that the first stimulating electrode contacts the broken bone or is located adjacent to the broken bone, the second stimulating electrode contacts the broken bone or is located adjacent to the bone broken, the broken area is located between the first stimulating electrode and the second stimulating electrode; and generating ultrasound toward the piezoelectric transducer via the skin of the body such that the stimulating electric current is generated and passes through the broken area.

Certain embodiments of the present disclosure provide a bone stimulation method for bone fracture healing of a broken bone in a body comprising: providing the bone stimulator described above, the bone stimulator being implanted in the body such that the first stimulating electrode contacts the broken bone or is located adjacent to the broken bone, the second stimulating electrode contacts the broken bone or is located adjacent to the bone broken, the broken area is located between the first stimulating electrode and the second stimulating electrode; and generating ultrasound toward the broken area and the piezoelectric transducer via the skin of the body such that the broken area is stimulated by the ultrasound and the stimulating electric current is generated and passes through the broken area.

Certain embodiments of the present disclosure provide a bone stimulation method for bone fracture healing of a broken bone in a body comprising: providing the bone stimulator described above, the bone stimulator being implanted in the body such that the bone fixation component contacts the broken bone, the second stimulating electrode contacts the broken bone or tissue adjacent to the broken area, the bone area is located between the bone fixation component and the second stimulating electrode; and generating ultrasound toward the piezoelectric transducer via the skin of the body such that the stimulating electric current is generated and passes through the broken area.

Certain embodiments of the present disclosure provide a bone stimulation method for bone fracture healing of a broken bone in a body comprising: providing the bone stimulation system described above, the bone stimulator and the bone fixation component being implanted in the body such that the first stimulating electrode contacts the broken bone, the second stimulating electrode contacts the broken bone or tissue adjacent to the broken bone, the broken area is located between the first stimulating electrode and the second stimulating electrode; and generating ultrasound toward the piezoelectric transducer via the skin of the body such that the stimulating electric current is generated and passes through the broken area of the bone fracture.

Certain embodiments of the present disclosure provide a bone stimulation method for bone fracture healing of a broken bone in a body comprising: providing the bone stimulation system described above; the bone stimulator and the bone fixation structure being implanted in the body such that the first stimulating electrode contacts the broken bone, the second stimulating electrode contacts the broken bone, the broken area is located between the first stimulating electrode and the second stimulating electrode; and generating ultrasound toward the piezoelectric transducer via the skin of the body such that the stimulating electric current is generated and passes through the broken area.

Certain embodiments of the present disclosure provide a bone stimulation method for bone fracture healing of a broken bone in a body comprising: generating ultrasound; converting power of the ultrasound into electric power; generating a stimulating electric current from the electric power; and passing the stimulating electric current through the broken area in the broken bone.

In certain embodiments, the bone stimulation method further comprises directing a portion of the ultrasound toward the broken area.

Certain embodiments of the present disclosure provide a bone stimulator for bone fracture healing of a broken bone in a body, the bone stimulator being implantable in the body and comprising: a piezoelectric transducer for converting mechanical power into electric power; a signal conditioning circuit for generating a stimulating electric current from the electric power; a first stimulating electrode for contacting or being located adjacent to the broken bone; and a second stimulating electrode for contacting the broken bone or being located adjacent to the broken bone, the first stimulating electrode and the second stimulating electrode being arranged such that the broken area in the broken bone is located between the first stimulating electrode and the second stimulating electrode such that the stimulating electric current passes through the broken area.

In certain embodiments, the mechanical power is acoustic power.

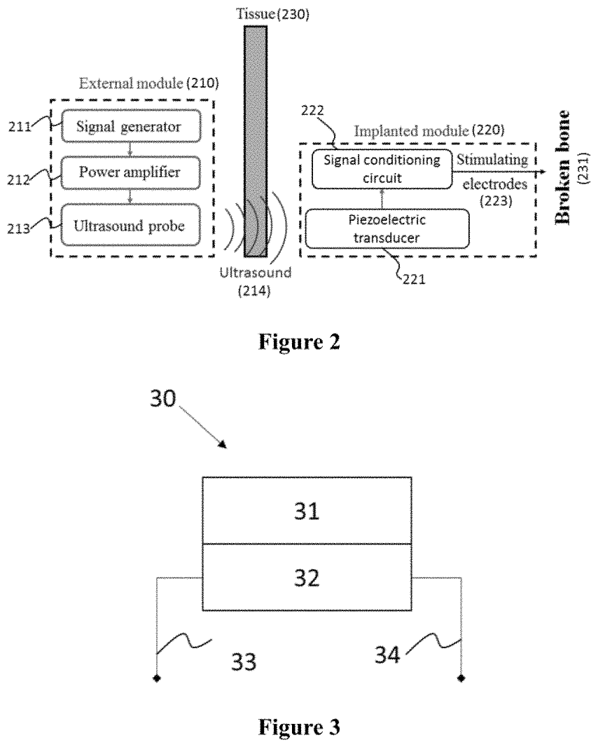

FIG. 2 depicts a bone stimulation system 200 for bone fracture healing of a broken bone. The bone stimulation system 200 comprises an external module 210 (that is an ultrasound generator) and an implanted module 220 (that is a bone stimulator). The external module 210 includes a signal generator 211, a power amplifier 212 and ultrasound probe 213. The signal generator 211 produces sinusoidal signal at one frequency from the range 0.5-20 MHz. The signal is then amplified by the power amplifier 212. The amplified signal serves as driving voltage for the ultrasound probe 213 connected to the power amplifier 212. The transducer of the ultrasound probe 213 generates ultrasound 214 in resonance frequency for one channel output of the external module 220. By selecting different frequency of the signal generator, different transducers of the ultrasound probe 213 generates different frequency of ultrasound signal for multichannel stimulation. The ultrasound probe 213 of the external module 210 can be attached to skin using ultrasound gel or other coupling liquid to facilitate the ultrasound 214 to pass through tissue 230.

The implanted module 220 comprises a piezoelectric transducer 221, a signal conditional circuit 222 and stimulating electrodes 223. The ultrasound 214 received by the piezoelectric transducer 221, which is incorporated in a bone fixation component, generates electric signal at its resonance frequency. The electric signal is then rectified and amplified by the signal conditioning circuit 222 to generate appropriate direct current signal for bone stimulation. The signal generator 211 can be controlled to generate different current amplitudes for bone stimulation. The stimulating electrodes 223 connect to a broken bone 231 for providing bone stimulation.

The bone stimulation system 200 requires minimal invasive procedure as the implanted module 220 is combined with the bone fixation component required for bone fracture healing such that two surgeries required to implant and explant the cathodes for treatment can be avoided.

FIG. 3 depicts a bone stimulator 30 according to certain embodiments. The bone stimulator 30 comprises a piezoelectric transducer 31, a signal conditioning circuit 32, a first stimulating electrode 33 and a second stimulating electrode 34. The piezoelectric transducer 31 converts power of ultrasound into electric power. The signal conditioning circuit 32 generates a stimulating electric current from the electric power. The first stimulating electrode 33 connects a first output of the signal conditioning circuit 32 to a broken bone, and the second stimulating electrode 34 connects a second output of the signal conditioning circuit 32 to the broken bone or tissue adjacent to the broken bone. The first stimulating electrode 33 and the second stimulating electrode 34 are arranged such that a broken area in the broken bone is located between the first stimulating electrode 33 and the second stimulating electrode 34 so as to circulate the stimulating electric current via the broken area.

The bone stimulator can be implanted in a body as a standalone implant or in combination with any other bone fixation components. In certain embodiments, the bone stimulator is configured to be biocompatible and tiny such that it can permanently stay in the body. In certain embodiments, the bone stimulator is, at least substantially, made from biodegradable materials such that an extra surgery for removing it from the body can be avoided.

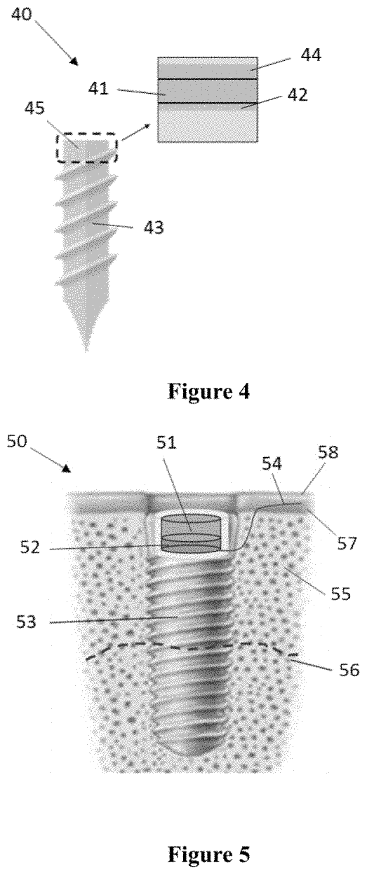

FIG. 4 depicts a screw-typed bone stimulator 40 according to certain embodiments. The screw-typed bone stimulator 40 comprises a piezoelectric transducer 41, a signal conditioning circuit 42, a metallic screw 43 and a coating layer 44. The metallic screw 43 is electrically connected to the signal conditioning circuit 42 and is used for being inserted into a broken bone such that the metallic screw 43 is a part of the stimulating electrode. The metallic screw 43 has a top hole 45, the piezoelectric transducer 41 and the signal conditioning circuit 42 are located within the top hole 45, and the top hole 45 is closed by the coating layer 44 to protect the piezoelectric transducer 41 and the signal conditioning circuit 42. The piezoelectric transducer 41 is located between the coating layer 44 and the signal conditioning circuit 42 such that ultrasound can easily arrive at the piezoelectric transducer 41 by passing through the coating layer 44 only.

FIG. 5 depicts a screw-typed bone stimulator 50 according to certain embodiments. The screw-typed bone stimulator 50 comprises a piezoelectric transducer 51, a signal conditioning circuit 52, a metallic screw 53 (i.e., a stimulating cathode) and a wire 54 (i.e., a stimulating anode). The piezoelectric transducer 51 is located on the signal conditioning circuit 52 located on the top of the metallic screw 53. The metallic screw 53 and the wire 54 are connected to the signal conditioning circuit 52. The metallic screw 53 is inserted into a broken bone 55 via a broken area 56 to connect and fix the broken bone 55 in place. The wire 54 is attached to tissue 57 adjacent to the broken bone 55 under a skin 58. As the broken area 56 is located between the metallic screw 53 and the wire 54, a stimulating electric current can be generated between metallic screw 53 and the wire 54 and pass through the broken area 56. As the piezoelectric transducer 51 is located between the skin 58 and the broken area 56, ultrasound generated by an ultrasound generator located above the skin 58 can also arrive at the broken area 56 for achieving ultrasound bone stimulation.

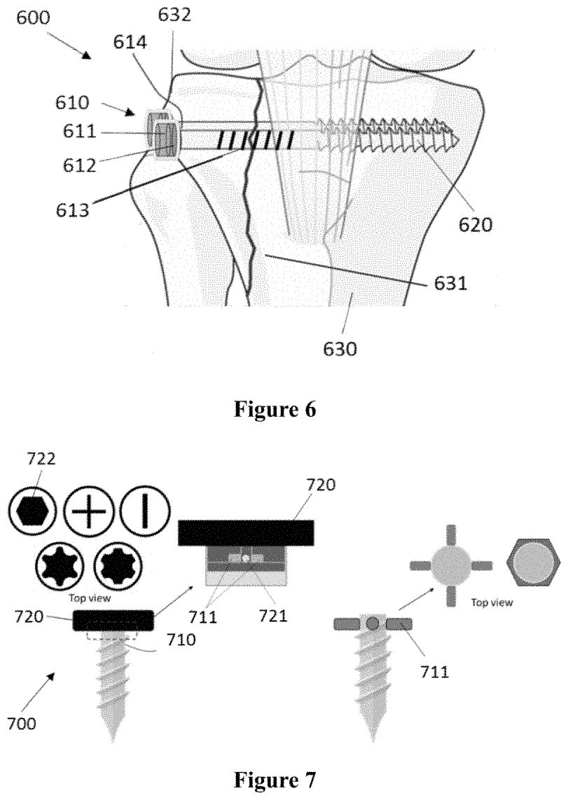

FIG. 6 depicts a bone stimulation system 600 according to certain embodiments. The bone stimulation system 600 comprises a bone stimulator 610 and a non-metallic screw 620. The bone stimulator 610 comprises a piezoelectric transducer 611, a signal conditioning circuit 612, a coiled cathode 613 and a wire anode 614. The non-metallic screw 620 is inserted into a broken bone 630 via a broken area 631 of a bone fracture. The piezoelectric transducer 611 and the signal conditioning circuit 612 are attached to the top of the non-metallic screw 620. The coiled cathode 613 coils around the non-metallic screw 620 and passes through the broken area 631. The wire anode 614 is attached to tissue 632 adjacent to the broken bone 630 such that a stimulating electric current can pass through the broken area 631.

FIG. 7 depicts a bone fixation component 700 according to certain embodiments. The bone fixation component 700 comprises a screw 710 and a removable cap 720 being attachable to the top of the screw 710 for inserting the screw 710 into a bone by a screwdriver. A bone stimulator can be embedded inside the screw 710. The screw 710 comprises four blocks 711 attached to the top of the screw 710. The removable cap 720 comprises four projections 721 located within the removable cap 720 for accommodating the four blocks 711 respectively. The removable cap 720 has slot 722 located on the removable cap 720 and fitted for a tip of the screwdriver. The removable cap 720 can be magnetic for facilitating the screwing with the screwdriver.

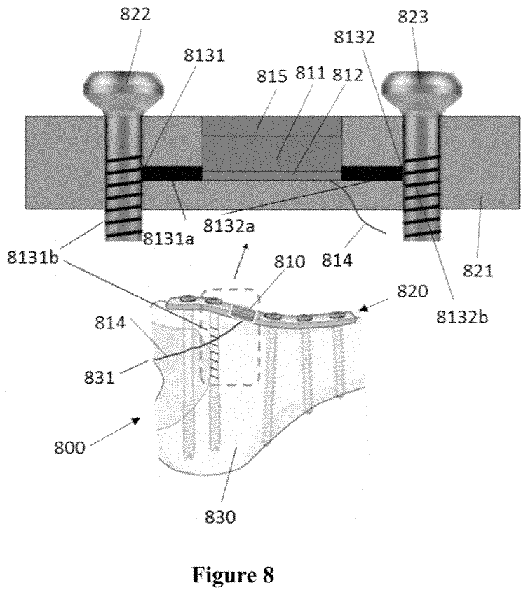

FIG. 8 depicts a bone stimulation system 800 according to certain embodiments. The bone stimulation system 800 comprises a bone stimulator 810 and a bone fixation structure 820. The bone stimulator 810 comprises a piezoelectric transducer 811, a signal conditioning circuit 812, two coiled cathodes 8131, 8132, a wire anode 814 and a coating layer 815. The bone fixation structure 820 comprises a bone fixation plate 821, a first screw 822 and a second screw 823. The bone fixation plate 821 is attached to a broken bone 830 for fixing the broken bone 830 in place. The first screw 822 and the second screw 823 connect the bone fixation plate 821 to the broken bone 830. The piezoelectric transducer 811 and the signal conditioning circuit 812 are embedded in the bone fixation plate 821 and closed by the coating layer 815. The coiled cathode 8131 has a connecting portion 8131a and a coiled portion 8131b. The connecting portion 8131a is embedded in the bone fixation plate 821 and the coiled portion 8131b coils around the first screw 822. The coiled cathode 8132 has a connecting portion 8132a and a coiled portion 8132b. The connecting portion 8132a is embedded in the bone fixation plate 821 and the coiled portion 8132b coils around the first screw 823. The wire anode 814 connects tissue adjacent to the broken bone 830 such that a broken area 831 of the broken bone 830 is located between the coiled portions 8131b, 8132b and the wired anode 814.

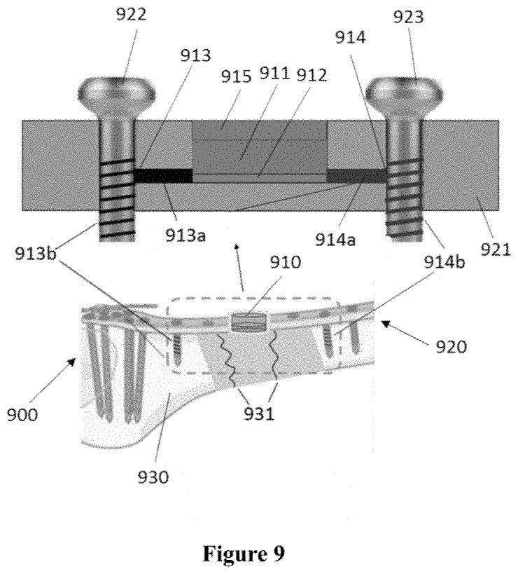

FIG. 9 depicts a bone stimulation system 900 according to certain embodiments. The bone stimulation system 900 comprises a bone stimulator 910 and a bone fixation structure 920. The bone stimulator 910 comprises a piezoelectric transducer 911, a signal conditioning circuit 912, a coiled cathode 913, a coiled anode 914 and a coating layer 915. The bone fixation structure 920 comprises a bone fixation plate 921, a first screw 922 and a second screw 923. The bone fixation plate 921 is attached to a broken bone 930 for fixing the broken bone 930 in place. The first screw 922 and the second screw 923 connect the bone fixation plate 921 to the broken bone 930. A broken area 931 of the broken bone 930 is located between the first screw 922 and the second screw 923. The piezoelectric transducer 911 and the signal conditioning circuit 912 are embedded in the bone fixation plate 921 and closed by the coating layer 915. The coiled cathode 913 has a connecting portion 913a and a coiled portion 913b. The connecting portion 913a is embedded in the bone fixation plate 921 and the coiled portion 913b coils around the first screw 922. The coiled anode 914 has a connecting portion 914a and a coiled portion 914b, and the connecting portion 914a is embedded in the bone fixation plate 921 and the coiled portion 914b coils around the second screw 923 such that the broken area 931 is located between the coiled portion 913b and the coiled portion 914b.

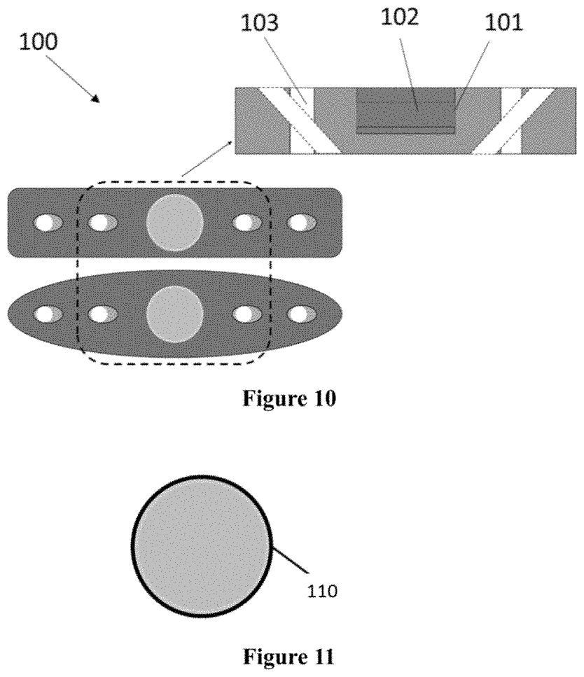

FIG. 10 depicts a bone fixation plate 100 according to certain embodiments. The bone fixation plate 100 comprises a hole 101 for accommodating a bone stimulator 102 and a plurality of through holes 103 for accommodating screws.

In certain embodiments, an image-guided method (ultrasound imaging) can be also used for monitoring the position of the bone stimulator inside a body. FIG. 11 depicts an acoustic absorber 110 for being located on the top of ultrasound receiving area of a bone stimulator so that the bone stimulator can be easily detected by ultrasonic imaging.

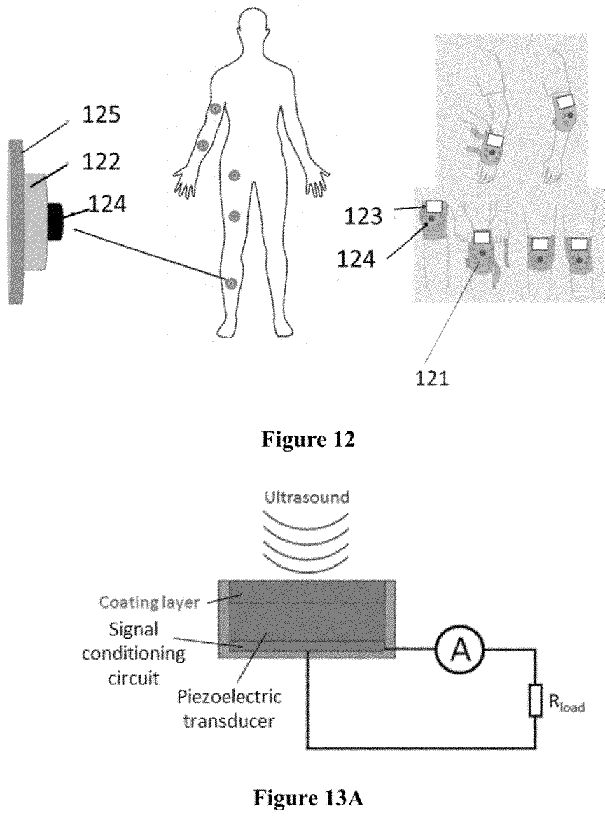

FIG. 12 depicts an ultrasonic generator embedded in a wearable protective gear 121 such that the ultrasonic generator can be located in different locations of a body. A single/multiple transducer 122 is connected with a power amplifier 123 in the wear protective gear 121 by a connector 124. The power amplifier 123 drives single/multiple transducer 122 to generate ultrasound power. A sticker 125 is used to fix single/multiple transducer 122 at the targeted area.

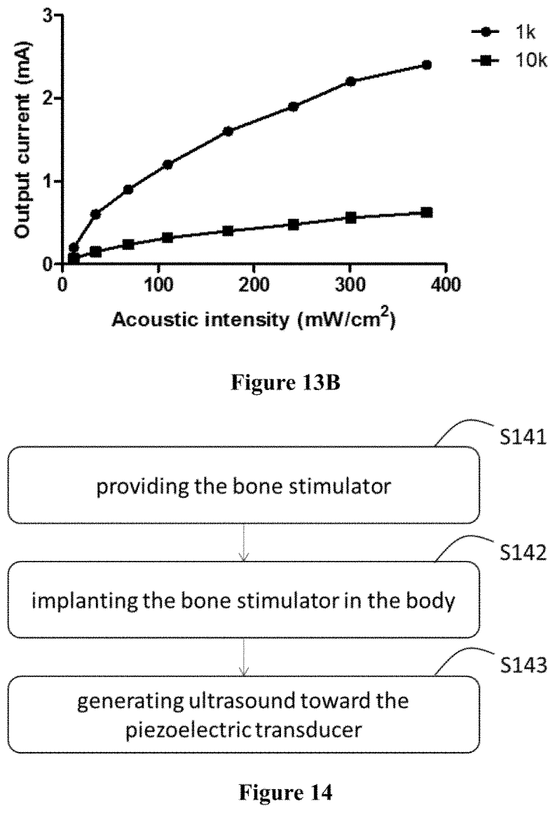

FIG. 13A depicts an experimental set-up for measuring a stimulating electric current generated by a bone stimulator. FIG. 13B shows output direct current of the bone stimulator at different ultrasound intensities. When the resistance is 10 kΩ, the output current increases from 0 mA to 0.6 mA following with the ultrasound intensity from 0 mW/cm² to 400 mW/cm². When the resistance is 1 kΩ, the output current increases from 0 mA to 2.3 mA following with the ultrasound intensity from 0 mW/cm² to 400 mW/cm².

FIG. 14 is a flow chart depicting a bone stimulation method for bone fracture healing of a broken bone in a body according to certain embodiments. In step S141, the bone stimulator described above is provided. In step S142, the bone stimulator is implanted in the body such that the first stimulating electrode contacts the broken bone, the second stimulating electrode contacts the broken bone or tissue adjacent to the broken bone, the broken area in the broken bone is located between the first stimulating electrode and the second stimulating electrode. In step S143, ultrasound is generated by an ultrasonic generator toward the piezoelectric transducer of the bone stimulator such that the stimulating electric current is generated and passes through the broken area of the bone fracture.

In certain embodiments, the bone stimulator is located between the ultrasonic generator and the broken area such that the ultrasound also arrives at the broken area for obtaining both of the electrical bone stimulation and ultrasound bone stimulation.

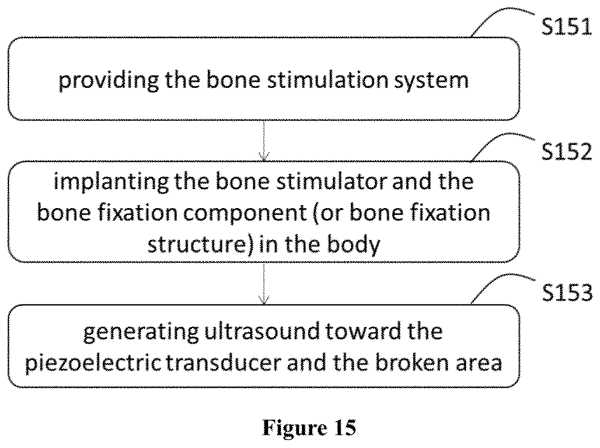

FIG. 15 is a flow chart depicting a bone stimulation method for bone fracture healing of a broken bone in a body according to certain embodiments. In step S151, the bone stimulation system described above is provided. In step S152, the bone stimulator and the bone fixation component (or bone fixation structure) are implanted in the body such that the first stimulating electrode contacts the broken bone, the second stimulating electrode contacts the broken bone or tissue adjacent to the broken bone, the broken area of the broken bone is located between the first stimulating electrode and the second stimulating electrode. In step S153, ultrasound is generated toward the piezoelectric transducer of the bone stimulator and the broken area such that the broken area is stimulated by the ultrasound and the stimulating electric current is generated and passes through the broken area.

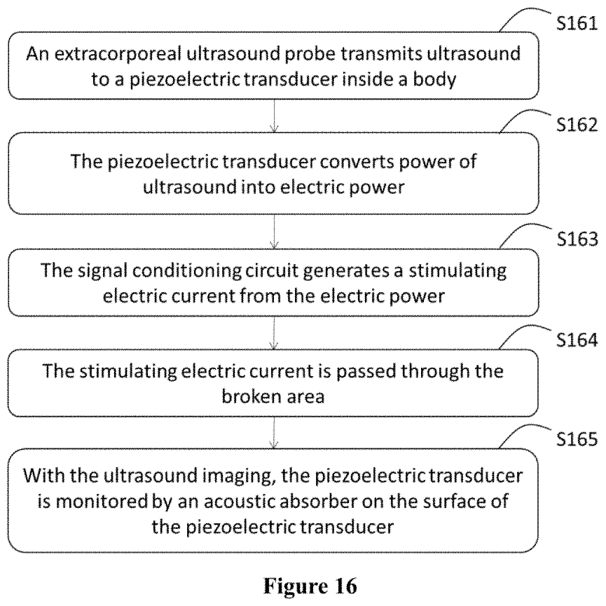

FIG. 16 is a flow chart depicting a bone stimulation method for bone fracture healing of a broken bone in a body according to certain embodiments. In step S161, an extracorporeal ultrasound probe transmits ultrasound to a piezoelectric transducer inside a body through skin. In step S162, the piezoelectric transducer converts power of ultrasound into electric power. In step S163, the signal conditioning circuit generate a stimulating electric current from the electric power. In step S164, the stimulating electric current is passed through the broken area. In step S165, with the ultrasound imaging, the piezoelectric transducer is monitored by an acoustic absorber on the surface of the piezoelectric transducer.

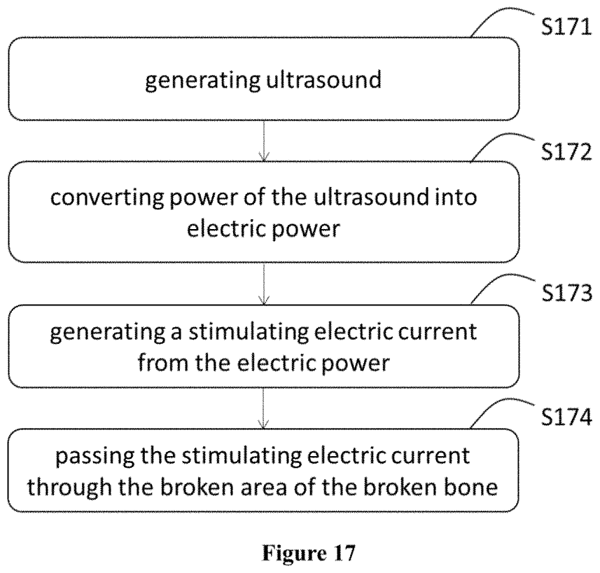

FIG. 17 is a flow chart depicting a bone stimulation method for bone fracture healing of a broken bone in a body according to certain embodiments. In step S171, ultrasound is generated. In step S172, power of the ultrasound is converted into electric power. In step S173, the electric power is converted into a stimulating electric current. In step S174, the stimulating electric current passes through the broken area in the broken bone.

In certain embodiments, the bone stimulation method further comprises directing a portion of the ultrasound toward the broken area.

In certain embodiments, the bone stimulation method for bone fracture healing is used via ultrasound signals with an image-guided method.

In certain embodiments, both of the electrical bone stimulation and ultrasound bone stimulation are used for bone fracture healing.

In certain embodiments, a flexible ultrasound probe or array included in the wearable external module is used to deliver ultrasound signal to the implanted module.

In certain embodiments, wireless image collection and remote image processing are used to monitor the implanted bone stimulator.

In certain embodiments, artificial intelligence method is used for optimizing the ultrasound intensity, duration, etc.

Thus, it can be seen that an improved apparatus and method have been disclosed which eliminates or at least diminishes the disadvantages and problems associated with prior art apparatus and methods. Accordingly, the present bone stimulation system is tiny, portable, wearable, as well as more accurate, durable and effective for fracture recovery of individuals. The present method provide a wireless method to power up the system for bone fracture healing. As the bone stimulator is a passive device such that no battery is required to be implanted into the body for avoiding battery replacement. In addition, the bone stimulator can be combined with the bone fixation component such that extra surgery for removing the bone stimulator from the body can be avoided. As ultrasound not only penetrate tissues to reach deep inside the body to generate sufficient electric current for electrical bone stimulation but also arrives at the broken area to have positive effects on bone healing, the combining effect from both electrical bone stimulation and ultrasound bone stimulation is obtained by the present method.

Although the invention has been described in terms of certain embodiments, other embodiments apparent to those of ordinary skill in the art are also within the scope of this invention. Accordingly, the scope of the invention is intended to be defined only by the claims which follow.

1. A bone stimulator for bone fracture healing of a broken bone in a body, the bone stimulator being implantable in the body and comprising:
a piezoelectric transducer for converting power of ultrasound into electric power;
a signal conditioning circuit for generating a stimulating electric current from the electric power;
a first stimulating electrode for contacting the broken bone or being located adjacent to the broken bone; and
a second stimulating electrode for contacting the broken bone or being located adjacent to the broken bone, the first stimulating electrode and the second stimulating electrode being arranged such that the broken area in the broken bone is located between the first stimulating electrode and the second stimulating electrode such that the stimulating electric current passes through the broken area.

2. The bone stimulator of claim 1, wherein the piezoelectric transducer, the signal conditioning circuit, the first stimulating electrode and the second stimulating electrode are biocompatible or biodegradable.

3. (canceled)

4. The bone stimulator of claim 1, wherein the piezoelectric transducer comprises a polymeric piezoelectric material or an inorganic piezoelectric material.

5. The bone stimulator of claim 1, wherein the piezoelectric transducer comprises lead zirconate titanate (Pb[ZrₓTi₁₋ₓ]O₃), lead titanate (PbTiO₃), Zinc oxide (ZnO), barium titanate (BaTiO₃) or polyvinylidene difluoride (PVDF).

6. The bone stimulator of claim 1, wherein each of the first stimulating electrode and the second stimulating electrode comprises copper, titanium, silver, or a carbon-based material.

7. The bone stimulator of claim 1 further comprising a coating layer or an enclosure for protecting the piezoelectric transducer and the signal conditioning circuit, and wherein the coating layer and the enclosure are biocompatible or biodegradable.

8. (canceled)

9. The bone stimulator of claim 7, wherein the coating layer and the enclosure comprise silicone, polytetrafluoroethylene, polydimethylsiloxane (PDMS), dimethyl silicone, or polyurethane.

10. The bone stimulator of claim 1, wherein the first stimulating electrode comprises a bone fixation component for contacting the broken bone and fixing the broken bone in place.

11. The bone stimulator of claim 10, wherein the bone fixation component is used for being inserted into the broken bone through the broken area.

12. The bone stimulator of claim 10, wherein the bone fixation component includes a screw, a pin, a nail, a rod, a panel or a plate.

13. The bone stimulator of claim 10, wherein the bone fixation component comprises a metallic material, a conductive biodegradable material, a conductive polymeric material, or a conductive ceramic material.

14. The bone stimulator of claim 10, wherein the bone fixation component comprises a hole, the piezoelectric transducer and the signal conditioning circuit being accommodated within the hole.

15. The bone stimulator of claim 14 further comprising a coating layer closing the hole.

16. The bone stimulator of claim 10, wherein the first stimulating electrode further comprises a connecting portion connecting the bone fixation component to the signal conditioning circuit.

17. The bone stimulator of claim 1, wherein the first stimulating electrode comprises a first bone fixation component for fixing the broken bone in place; and the second stimulating electrode comprises a second bone fixation component for fixing the broken bone in place.

18. The bone stimulator of claim 1, wherein the stimulating electric current is direct current in a range of 1 μA to 30 mA.

19. The bone stimulator of claim 1, wherein the waveform and the magnitude of the stimulating electric current is controlled by an external ultrasound generator.

20. (canceled)

21. A bone stimulation system for bone fracture healing of a broken bone comprising:
the bone stimulator of claim 1; and
a bone fixation component for fixing the broken bone in place, the first stimulating electrode being attached to the first bone fixation component,
wherein the bone fixation component is electrically non-conductive.

22-26. (canceled)

27. A bone stimulation system for bone fracture healing of a broken bone comprising:
the bone stimulator of claim 1; and
a bone fixation structure comprising a bone fixation plate a first bone fixation component and a second bone fixation component,
the bone fixation plate being attached to the broken bone for fixing the broken bone in place, the first bone fixation component connecting the bone fixation plate to the broken bone, the first stimulating electrode being attached to the first bone fixation component,
the second bone fixation component connecting the bone fixation plate to the broken bone, the second stimulating; electrode being attached to the second bone fixation component.

28-31. (canceled)

32. A bone stimulation method for bone fracture healing of a broken bone in a body comprising:
providing the bone stimulator of claim 1, the bone stimulator being implanted in the body such that the first stimulating electrode contacts the broken bone or tissue adjacent to the broken area, or is located adjacent to the broken bone, the second stimulating electrode contacts the broken bone or is located adjacent to the bone broken, the broken area is located between the first stimulating electrode and the second stimulating electrode; and
generating ultrasound toward the piezoelectric transducer and/or the broken area via the skin of the body such that the stimulating electric current is generated and passes through the broken area.

33-40. (canceled)
